(19) United States
(12) Patent Application Publication
(10) Pub. No.: US 2016/0243244 A1
(43) Pub. Date: Aug. 25, 2016

(54) METHOD AND SYSTEM FOR ALLEVIATING JOINT PAIN

(71) Applicant: EmboMedics, Inc., Maple Grove, MN (US)

(72) Inventors: Jafar Golzarian, Plymouth, MN (US); Lihui Weng, Woodbury, MN (US); Omid Souresrafil, Maple Grove, MN (US)

(21) Appl. No.: 15/048,487

(22) Filed: Feb. 19, 2016

Related U.S. Application Data
(60) Provisional application No. 62/120,028, filed on Feb. 24, 2015.

(51) Int. Cl. A61K 47/38; A61K 31/192; A61K 47/36; A61K 9/00

(52) U.S. Cl. CPC A61K 47/38; A61K 9/0019; A61K 31/192; A61K 47/36

(57) ABSTRACT

A system for providing a drug-eluting resorbable hydrogel for treating chronic pain, the system including carboxymethyl chitosan and oxidized carboxymethyl cellulose solutions, which together with one or more bioactive agents, can be mixed and delivered at the time of use in order to gel in situ within a joint space. Once delivered, the hydrogel can provide bioactive agents such as NSAIDs directly to the areas commonly associated with chronic pain, including to the spine, knee, shoulder, and elbow. The hydrogel is substantially non-inflammatory, and substantially biodegradable over time, to provide prolonged drug release.

### FIELD OF THE INVENTION

The present invention provides a composition and corresponding kit and method of sustained, localized drug delivery for use in treating joint inflammation. The composition includes the use Of an active agent, e.g., such as ibuprofen, in combination with a crosslinked hydrogel.

### BACKGROUND

A large percentage of the population experiences chronic pain, including spinal and joint pain related to diseases such as osteoarthritis and rheumatoid arthritis, stresses from sports injuries, work conditions, accidents, and aging. Common pain management interventions include over-the-counter analgesics such as acetaminophen and non-steroidal anti-inflammatory drugs (NSAIDs), as well as other medications such as duloxetine and, in more severe cases, opioids. However, when taken orally there is a risk of gastrointestinal and cardiovascular side effects that discourage their long-term use. Oral medication requires higher doses of drug, and even daily dosing is inconvenient to the patient, with a high probability of noncompliance. Topical NSAIDs such as ibuprofen appear to be efficacious for local delivery of pain relief, but still have the disadvantage of requiring application several times daily. For joint or spinal pain, localized interventions such as intra-articular steroid injections provide only short-term relief (1-6 weeks) that limits their utility and requires frequent visits to a healthcare provider. Epidural corticosteroid injections for back pain now carry a warning from the Food and Drug Administration (FDA). Thus, there is an unmet need for safe, convenient, and long-term relief of joint and spinal pain.

Potential injectable strategies have explored microspheres and microcapsules, nanoparticles, and hydrogels. While microspheres have shown promise, it has been challenging to identify chemical compositions that minimize burst release (e.g., rapid drug elution immediately after injection). Early attempts using formulations based on biodegradable poly(lactide) glycolide copolymers (PLGA) had considerable burst release of ibuprofen, and usually required a carrier material such as fibrin glue or Microfil brand compounds. Recent results are promising in terms of reducing burst release and achieving sustained ex vivo release of ibuprofen, but exhibited various potential shortcomings as well.

Applicant has particular expertise with respect to drug loaded hydrogels and microspheres for embolization of hypervascularized and cancerous tumors and arthritic pain. See for instance. US 20140171907 (Liquid hydrogel material including carboxymethyl chitosan crosslinked with carboxymethyl cellulose); US 20140099374 (Bioresorbable Embolization Microspheres); US 20110082427 (Bioresorbable Embolization Microspheres); EP2485777 (Bioresorbable Embolization Microspheres); and AU 2010303552 (Bioresorbable Embolization Particles), the disclosures of each of which are incorporated herein by reference.

### SUMMARY

Applicant has developed a system, including deliverable composition and corresponding method, for localizing the delivery of pain medication to one or more areas where it is needed, bypassing the gastrointestinal tract and thereby reducing the risk of GI and cardiovascular adverse reactions, as well as lowering the overall dose of medication. In a preferred embodiment, the present system provides a system for intraarticular drug delivery in a manner that obviates the need for patients to take medication daily or even several times a day, thereby increasing patient quality of life. In turn, a preferred system can provide treatment that is preferably safer and longer lasting than current injectable solutions, such as steroids. Overall, it preferably improves pain management, while also leading to a lower incidence of physical and mental comorbidities often associated with chronic pain, with consequent reduction in treatment and medical costs.

Applicant has developed drug-eluting resorbable hydrogels that are well-suited to treating chronic pain. They are in situ gelable, injectable, and drug loadable, having physicochemical properties that are easily modulated. Thus, these hydrogels can be used to deliver anti-inflammatory drugs such as NSAIDs directly to the areas commonly associated with chronic pain, including to the spine, knee, shoulder, and elbow. In contrast to other direct-delivery technologies, the components of this hydrogel can be naturally derived, non-inflammatory, and substantially biodegradable. In addition, the rate of resorption of the hydrogel can be controlled by adjusting crosslinking density, thus providing the desired duration of localized pharmacological effect. Moreover, a preferred hydrogel formulation can be prepared with minimal technological know-how or equipment, which is ideal for a wide array of healthcare providers. It can be designed to gel in situ, allowing it to conform to and substantially fill an anatomical space, for instance, in contrast to PLGA microspheres that require a carrier (such as fibrin glue or Microfil brand silicone rubber injection compounds) to retain them in place.

In a preferred embodiment, the system comprises the preparation and use of a composition that comprises a) hydrogel and b) active agent, in combination with a delivery device adapted to deliver the composition to a joint space. Suitable active agents include, but are not limited to, glucocorticoids (e.g., betamethasone, dexamethasone, prednisolone, rimexolone, and triamcinolone, including salts thereof), anesthetics (e.g., lidocaine), NSAIDs (e.g., aspirin, Choline and magnesium salicylates, Celecoxib, Diclofenac, Diflunisal, Etodolac, Fenoprofen calcium, Flurbiprofen, Ibuprofen, Indomethacin, Ketoprofen, Magnesium salicylate, Meclofenamate sodium, Mefenamic acid, Meloxicam, Nabumetone, Naproxen, Naproxen, Oxaprozin, Piroxicam, Rofecoxib, Salsalate, Sodium salicylate, Sulindac, Tolmetin sodium, and Valdecoxib. Preferred active agents provide an optimal combination of properties, including efficacy in vivo, compatibility with the system and hydrogel of this invention, including the ability to be sterilized, and stored.

A system of this invention can provide various advantages associated with sustained localized drug delivery. A preferred composition of this invention requires minimal storage and processing criteria, and can be easily used with existing technical know-how and procedures for mixing and delivering hydrogel compositions. A preferred composition is also preferably resorbable in situ, and possesses desirable drug-eluting characteristics that can be maintained over a period of weeks or months, having minimal risk of injection site reactions or other adverse effects. In a particularly preferred embodiment, the drug composition is delivered by means of intra-articular injection in order to be localized within the cartilage, which is typically not vascularized. The system permits the drug to remain in situ, thereby minimizing the extent to which drug might enter the systemic circulation.

### DETAILED DESCRIPTION

In one embodiment, the present invention provides a system for alleviating pain within an orthopedic joint space, the system comprising: a) a deliverable composition comprising a plurality of parts adapted to be mixed at the time of use in order to provide a deliverable composition adapted to form a hydrogel in situ within the joint space, b) one or more active agents adapted to be included within the deliverable composition, and c) a device adapted to mix the composition parts, including active agent(s), in order to deliver the deliverable composition to the joint space.

In a preferred embodiment, the plurality of parts comprises a first solution comprising between about 0.5% and about 3% weight per volume (w/v) carboxymethyl chitosan (CCN) in a first solvent, and a second solution comprising between about 0.5% and about 3% w/v oxidized carboxymethyl cellulose (OCMC) in a second solvent, and the first and second parts are mixed to form a hydrogel precursor material in the form of a single liquid phase that can be delivered to the joint space in order to form a hydrogel the targeted joint site.

Applicant has developed a novel approach to deliver one or more active agents, e.g., for pain relief, directly to areas of joint and spinal pain. The method and corresponding system involve prolonged, slow release drug delivery, and can be used to reduce interventions to any desired period, e.g., once every few months, thereby affording pain sufferers long-term relief and enhanced quality of life without frequent visits to their healthcare practitioner. In addition, by delivering pain relief directly and specifically to the site itself, a preferred system can minimize cardiovascular and gastrointestinal side effects. In contrast to other direct-delivery technologies, some preferred compositions can be used to release drug in a controlled manner that does not involve an initial and undesired burst of drug release. In so doing, the current system can minimize or avoid current limitations associated with the use of localized injections, using current commercial products, which are typically designed to provide relatively short term relief. By contrast, Applicant has found the manner in which such limitations can be addressed, and potentially overcome, by the use of an extended-release drug-eluting hydrogel in the manner presently described and claimed.

In contrast to compounds such as PLGA, as used in other therapeutic applications, which degrades extremely slowly while producing acidic byproducts that cause inflammation, the hydrogel of the present invention will preferably comprise naturally derived cellulose and chitosan, both of which can eventually be metabolized, degraded, and/or substantially cleared from the joint space over time (e.g., weeks to months).

A composition of the present invention provides added benefits, given its targeted nature, since the hydrogel is substantially, and preferably completely, bioresorable with no synthetic ingredients. In one such preferred embodiment, the system of this invention can provide an analgesic approach that requires only three or four injections (i.e., patient visits) per year.

A system of the present invention relies on the use of an in situ forming resorbable hydrogel. In a particularly preferred embodiment, the hydrogel eventually releases substantially all of the drug, leaving little if any detectable residue. A preferred material is substantially non-inflammatory and therefore less likely to result in injection site reactions and other adverse events. The word “substantially” as used in the present description, will generally refer to an extent sufficient for its intended use.

In a particularly preferred embodiment, a system of this invention provides a medication-loaded hydrogel composition for use in outpatient settings, which includes an in situ forming hydrogel that is capable of loading and releasing ibuprofen sodium salt. The hydrogel itself is capable of undergoing degradation under physiological pH in the presence of lysozyme, thereby releasing ibuprofen in a sustained manner, and rendering the hydrogel itself substantially resorbable in vivo.

Those skilled in the art, given the present description, will be able to determine various aspects associated with a composition of this invention, for instance, by establishing initial dosing guidelines based on the pharmacokinetics/pharmacodynamics (PK/PD) in appropriate models, including different species, including through the use of human clinical studies.

Applicant has developed a series of drug-eluting resorbable hydrogels that are loaded with drug and have physicochemical properties that are easily modulated. The hydrogels are comprised by mixing partially oxidized carboxymethyl cellulose (OCMC) and carboxymethyl chitosan (CCN) aqueous solutions. The rate of resorption of the hydrogel can be controlled by various means that will become apparent to those skilled in the art, including by adjusting crosslinking density, thus providing the desired duration of localized pharmacological effect. In preliminary experiments, the dry weight of the hydrogel showed a consistent decrease over the degradation period in both 4 mg/mL and 10 μg/mL lysozyme solutions. Drug release is sustained due to the ampholytic nature of CCN, which allows it to bind with ibuprofen and then slowly release the drug. This hydrogel formulation can be prepared at the time of use with existing technological know-how and equipment, and in a manner well suited for a wide array of healthcare providers. It gels in situ, allowing it to conform to, and substantially fill, anatomical spaces. Thus, it lends itself well to delivering NSAIDs, etc. directly into spine and large joints (e.g., knee, shoulder, or elbow) for slow-release drug delivery.

A hydrogel of the present invention can be delivered to a joint space using any suitable means, e.g., using needle-syringe techniques. See, for instance, “Intraarticular Drug Delivery in Osteoarthritis”, Gerwin, et al., Advanced Drug Delivery Reviews. 58(2006) 226-242, the disclosure of which is incorporated by reference. In a preferred embodiment, careful sterile technique is used avoid joint infection. Proper needle placement should be ensured, e.g., by means of radiographic or ultrasound techniques. Aspiration of the joint space (e.g., synovial fluid) at the time of injection can be associated with improved accuracy of injection, lessening of potential dilution effects, and the localization of hydrogel in situ, while the aspirated fluid can itself be used for diagnostic purposes.

The direct delivery of drug to a joint (e.g., knee, hand, and foot joints) also offers the possibility to achieve useful (e.g., therapeutic) drug concentrations at the site by applying low amounts of drug. A system of the present invention permits relatively few injections to be used over the course of several months to a year, which is particularly desirable given the potential for pain and infection. Gerwin et al. itself confirms “the need for the development of sustained release formulations, which support the continuous release of the drug from a depot in the joint space over a period of weeks or months”.

In a particularly preferred embodiment, a deliverable hydrogel of this invention provides an optimal combination of properties that include the ability to be prepared, sterilized (e.g., by autoclaving or filtration), stored and used in a suitable manner, and upon delivery and use, the compatibility of the hydrogel with physiological conditions at the joint site (e.g., including formulations that are isotonic, having a pH at or near that of the synovial fluid, and suitable stability in situ).

The present invention provides hydrogel materials comprising carboxymethyl chitosan (CCN) crosslinked with oxidized carboxymethyl cellulose (OCMC). The hydrogel materials described herein are delivered to a target region in liquid form and gel in situ (e.g., in vivo). For example, CCN is dissolved in a first solvent and OCMC is dissolved in a second solvent. The first and second solvents (including the CCN and the OCMC) are mixed at the time of injection or just before delivery to the target region. The CCN and the OCMC begin crosslinking (forming a hydrogel) upon mixing, but the crosslinking reaction does not complete instantaneously. Hence, the liquid mixture in which the crosslinking reaction is occurring can flow to the target region (joint space) prior to completion of the crosslinking reaction. The time from initial mixing to substantial completion of the crosslinking reaction can be influenced by the concentration of CCN and OCMC in the respective first and second solvents.

In some examples, one or both of the first and second solvents can include at least one additional component. For example, the first and/or second solvents can include contrast and/or at least one drug (e.g., pharmaceutical). In this way, the hydrogel are used to deliver drugs to a location in order to remain positioned within the location and/or are used as a radiopaque marker. In some embodiments, the crosslinking reaction between the CCN and OCMC can proceed without use of a small molecule crosslinking agent that might have the potential for cytotoxicity. Because of this, the hydrogel is expected to be biodegradable and biocompatible.

CCN is substantially non-toxic and biodegradable. Chitosan breaks down in the body to glucosamine, which are substantially completely absorbed by a patient's body. Similarly, CMC is substantially non-toxic and biodegradable. Thus a crosslinked polymer formed by CCN and OCMC is expected to be substantially non-toxic (i.e., biocompatible) and biodegradable (or bioresorbable), to the point where it is eventually metabolized by and/or cleared from the body.

The degree of oxidation of the CMC can be affected by, for example, the molar ratio of NaIO₄ to CMC repeating units. In some embodiments, the molar ratio of NaIO₄ molecules to CMC repeating units are between about 0.1:1 and about 0.5:1 (NaIO₄:CMC). The CMC can include a weight average molecular weight of between about 50,000 daltons (Da; equivalent to grams per mole (g/mol)) and about 800,000 Da. In some embodiments, a weight average molecular weight of the CMC is about 700,000 Da.

CCN are prepared by reacting chitosan to attach —CH₂COO⁻ groups in place of one of the hydrogen atoms in an amine group or a hydroxyl group. The reactant supplying the —CH₂COO⁻ can include, for example, monochloroacetic acid. Similar to oxidation of CMC, the extent of the addition of the —CH₂COO⁻ can affect the crosslink density when the CCN is reacted with the partially oxidized CMC to form the hydrogel.

Once the OCMC and the CCN have been prepared, each is mixed in a respective amount of a solvent, such as water (e.g., distilled water), saline, or PBS. For example, 0.1 milligram (mg) of OCMC can be mixed in 5 milliliter (mL) of water to form a first 2% weight/volume (w/v) solution. Similarly, as an example, 0.1 mg of CCN can be mixed in 5 mL of water to form a second 2% w/v solution. The solvent used in the OCMC solution can be the same as or different than the solvent used in the CCN solution.

In some examples, one or both of the OCMC solution or the CCN solution can include additional components. For example, either or both of the solutions can include at least one drug (e.g., pharmaceutical) and/or contrast. The contrast can be mixed into the solvent (e.g., water, saline, PBS, or the like), at any desired concentration, for instance, to between about 10% (volume of contrast/volume of solvent; v/v) and about 50% v/v, such as, for example, about 20% v/v.

Similarly, at least one drug is mixed in o the OCMC solution and/or the CCN solution at any concentration. In some examples, the concentration of drug is defined based on the dry composition (e.g., the composition excluding the solvent and contrast). In some examples, the drug is mixed into the hydrogel in a ratio of up to about 30% drug to dried polymer (on a weight basis; w/w), such as, for example, between about 5% w/w and about 30% w/w, or between about 5% w/w and about 20% w/w, or about 10% w/w, or about 20% w/w. The amount of drug can be determined so as to provide with a desired dosage, e.g., about 5 to about 15 mg/kg body weight.

The solutions can be provided (e.g., prepared, sterilized, stored) in any suitable container. In an example, the OCMC solution and the CCN solution are disposed in separate syringes. As described below, the syringes can facilitate dispensing of the solutions for mixing of the solutions and/or introduction of the solutions.

A preferred method of this invention also includes mixing the OCMC solution and the CCN solution to form the liquid hydrogel material. In some examples, the solutions are not mixed until shortly before introduction of the liquid hydrogel material into a body of a patient. The time at which the OCMC solution and the CCN solution are mixed is determined, at least in part, on the absolute and relative concentrations of OCMC and CCN in their respective solutions. Upon mixing the OCMC solution and the CCN solution, the two begin to react in order to form a hydrogel. In particular, an amino group on the CCN can react with an aldehyde group on the OCMC to form a Schiff base (i.e., an N═C double bond) and crosslink the CMC and the CCN.

In some examples, the crosslinking reaction between OCMC and CCN can proceed under relatively benign conditions. For example, the crosslinking reaction can be carried out at ambient pressures and ambient temperatures (e.g., a temperature within the body of the patient). In some embodiments, a portion of the reaction, e.g., before introduction of the liquid hydrogel material into the body of the patient, is carried out at a temperature above ambient, such as, for example, 50° C. Hence, in some examples, a first portion of the reaction is carried out at a first temperature and/or pressure and a second portion of the reaction are carried out at a second temperature and/or pressure. Exemplary ranges of temperatures in which the crosslinking reaction are performed include between about 20° C. and about 70° C., and at about 50° C. and/or about 37° C.

An extent of crosslinking between molecules of OCMC and CCN can affect mechanical properties of the resulting hydrogel. For example, a greater crosslinking density generally can provide greater mechanical strength (e.g., fracture strain), while a lower crosslinking density can provide lower mechanical strength. The crosslinking density can also affect the degradation rate of the hydrogel. For example, a greater crosslinking density can lead to a longer degradation time, while a lower crosslinking density can lead to a shorter degradation time. In some examples, the hydrogel can degrade through hydrolyzing of the C═N double bond.

The OCMC and CCN solutions can be mixed using any suitable technique. One technique for mixing the OCMC solution and the CCN solution includes mixing the OCMC solution and the CCN solution in a container. Another technique for mixing the OCMC solution and the CCN solution includes coupling a first syringe containing the OCMC solution and a second syringe containing the CCN solution to a three-way stopcock valve and mixing the solutions between the two syringes. The OCMC solution and the CCN solution also are mixed using other techniques. The active agent can be provided in any suitable manner, e.g., together with either or both solutions, or as yet another solution adapted to also be mixed prior to or at the time of hydrogel formation and/or delivery. In an optional embodiment, a hydrogel of this invention can be substantially prepared in vitro, in order to be delivered to the body, using suitable means, during or upon gelling. Similarly, a hydrogel of this invention can include the use of one or more other parts or layers, e.g., additional layers of the same or different materials, and/or the inclusion of suitable materials such as matrices in order to provide additional or different features, such as conformation or physical properties (e.g., rigidity, compressibility, and surface characteristics).

The OCMC and CCN solutions also are mixed in any ratio. In some examples, the OCMC solution and the CCN solution are mixed in a 1:1 ratio, such that equal volumes of OCMC solution and CCN solution are mixed. In other examples, the OCMC solution and the CCN solution are mixed in a ratio other than 1:1, such that is more OCMC solution or more CCN solution in the hydrogel precursor mixture. Because the CCN solution and the OCMC solution are mixed, the resulting concentrations of OCMC and CCN in the hydrogel precursor mixture are lower than the concentration of OCMC in the OCMC solution and the concentration of CCN in the CCN solution. The change in concentration will depend on the respective amounts of OCMC solution and CCN solution, and the concentration of OCMC in the OCMC solution and the concentration of CCN in the CCN solution.

After mixing OCMC solution and the CCN solution to form the hydrogel precursor mixture, the mixture can be introduced into a body of a patient. In some instances, the mixture is introduced into the body of the patient using a device (e.g., syringe or microcatheter). The device can be provided in any suitable manner, including in any of a variety of sizes. For instance, a hydrogel precursor mixture formed from mixing a 2% w/v OCMC solution and a 2% w/v CCN solution can be introduced through a syringe having an outer diameter of 3 French (Fr) (and an inner diameter of about 0.027 inch (about 0.6858 millimeter; mm)). In other examples, hydrogel precursor mixtures are introduced using syringes having smaller inner diameters (e.g., 1 Fr or 2 Fr), or having larger inner diameters (e.g., greater than 3 Fr).

In some examples, the location at which the hydrogel precursor mixture is introduced into the body of the patient relative to the targeted joint site is selected based at least in part on the concentration of the OCMC and CCN in the hydrogel precursor mixture. For example, hydrogel precursor mixtures with higher concentrations OCMC and CCN can form a gel more rapidly than hydrogel precursor mixtures with lower concentrations of OCMC and CCN. Accordingly, hydrogel precursor mixtures with higher concentrations of OCMC and CCN can be introduced nearer to the targeted joint site than hydrogel precursor mixtures with lower concentrations of OCMC and CCN (other conditions being the same or substantially similar). Hydrogel precursor mixtures with lower concentrations of OCMC and CCN can pass through the needle easier. Similarly, the total amount of hydrogel precursor mixture that is introduced into the body of the patient can be selected based at least in part on a size of the targeted joint site. For example, a joint site having a smaller space can require less hydrogel than a joint site having a larger space.

In some examples, the liquid hydrogel material can provide in the form of a kit. The kit can include a first mixture of OCMC and a first solvent in a first container and a second mixture of CCN and a second solvent in a second container. As described below, the solvent can include, for example, water, saline, or phosphate buffered saline (PBS). In some instances, the kit can additionally include a mixing device, such as a three-way stopcock, and/or an introduction device, such as a needle, a microcatheter, or the like. The kit can facilitate mixing of the first mixture and the second mixture to initiate the reaction between the OCMC and the CCN, followed by introduction of the liquid hydrogel material to a selected location of a body of a patient.

OCMC aqueous solutions were prepared in the manner described in Applicant's US Publication No. 20140171907 having OCMC concentrations of 1.5% w/v (grams OCMC per mL solvent), 1.8% w/v, or 2% w/v. CCN aqueous solutions having substantially the same concentrations were also prepared. A hydrogel was prepared by mixing in a 1:1 ratio OCMC and CCN aqueous solutions of similar concentrations OCMC and CCN (e.g., mixing equal volumes of a 1.5% w/v OCMC solution and a 1.5% w/v CCN solution). Once mixed, the hydrogel precursor mixtures were added into 24-well non-tissue culture plates and incubated at 37° C. for 2 hours to reach full gelation.

The gelation time of the CCN/OCMC precursor, with or without contrast (Optiray 320, available from Mallinckrodt, Inc., Hazelwood, Mo.) was determined by mixing about 100 μL of OCMC solution and about 100 μL CCN solution with a magnetic stir bar in a Petri dish (available from Becton, Dickinson and Company (BD), Franklin Lakes, N.J.) at 155 revolutions per minute (rpm) using a hotplate/stirrer (Isotemp 11-100, available from Fisher Scientific, Pittsburgh, Pa.). The gelation time was determined when the mixture formed a globule. The experiments were repeated four times per sample.

The morphology of lyophilized hydrogel was evaluated by scanning electron microscopy (SEM) (JEOL JSM-6700F, available from JEOL Ltd., Tokyo, Japan) after spray-coating the lyophilized hydrogel with gold. Swollen gel pieces were snap-frozen in liquid nitrogen and then lyophilized. A section of the dried gel was mounted on a metal stub coating a layer of conductive adhesive. SEM images were obtained at a 2.0 kV (kilovolt) acceleration voltage in a deceleration mode under a nitrogen atmosphere.

SEM revealed interconnecting pores in the hydrogel prepared from a 2% , w/v hydrogel precursor mixture (concentrations based on the OCMC solution and CCN solution before mixing) and pore size analysis gave an average pore diameter of 17±4 μm (mean±SD), demonstrating the possibility of drug diffusion or nutrition exchange through these pores. In addition, it was also observed that the pore size distribution depends on the swollen state of the hydrogel and polymer concentration (data not shown).

### EXAMPLES

A resorbable hydrogel is prepared by oxidizing carboxymethyl cellulose (CMC) with sodium periodate to form OCMC, and carboxymethyl chitosan (CCN) is generated by modifying chitosan with monochloroacetic acid. Crosslinking occurs by means of the reaction of —CHO groups on OCMC with free —NH₂ groups on CCN.¹⁷ To generate an ibuprofen loaded hydrogel (“ILH”), the OCMC/CCN precursor is mixed with ibuprofen (IBU) sodium salt before the gelation step. In addition, the ILH contains 20% (v/v) contrast media.

All experiments are performed using adult male Sprague-Dawley rats weighing 150-220 g and are performed with ethics committee approval using conventional techniques, including those for the induction of osteoarthritis (“OA”). Studies are performed in the manner outlined in Table 1.

TABLE 1

| Group | Animals | Treatment | Assessments |
|---|---|---|---|
| 1a | OA | ILH (left knee), | Joint tissue |
| | (both knees) | blank hydrogel | assessment |
| | | (right knee) | Hydrogel resorption |
| | | | Drug plasma |
| | | | concentration |
| 1b | OA | ILH (right knee) | IBU concentration in joint |
| 1c | (right knee) | IBU in saline (right | |
| | | knee) | |
| 2d | | None | Therapeutic efficacy |
| 2e | | ILH (right knee) | |
| 2f | | IBU in saline (right | |
| | | knee) | |

The resorption of ILH is determined at day 0 (N=3), or at 3 days, 1 week, 3 weeks, and 6 weeks (N=4 each time point), using conventional techniques. Left and right knee joints are removed and fixed in formalin and decalcified in 10% formic acid with repeated changes. After decalcification, the tissues are embedded in paraffin wax, sectioned at 3-5 μm, and stained with haemotoxylin and eosin. ILH resorption is assessed histologically, comparing the tissue sections at the time points shown in Table 2 with baseline (day 0). Tissue sections are also used to assess the safety of ILH injected into knee joints by histologic observation for inflammation, organization, capillary formation, reaction to foreign bodies, and fibrosis. A suitable grading scale can be used to determine the degree of inflammation: (1) mild: scant, scattered inflammatory cellular infiltration; (2) moderate: attenuated, patchy inflammatory cellular infiltration; or (3) marked: diffuse inflammatory cellular infiltration. In addition, clinical response is evaluated by a veterinarian blinded to the type of treatment through a physical exam of the rats for 3 days following injection, and then weekly until the animals are sacrificed.

The same rats are sampled to deter line IBU plasma concentration. Plasma is prepared from blood samples collected from the jugular vein at the time points indicated in Table 2. Following centrifugation at 4000 rpm for 15 min, the supernatants are collected and stored at −20° C. for drug analysis by HPLC.

To determine the concentration of IBU in joint tissue, rats in groups B and C (Table 1) are euthanized according to the schedule in Table 2 (N=4 for ILH rats, N=2 for IBU/saline rats). Joint tissue (cartilage and synovial membrane) is removed from the injected knee and homogenized in 0.5 mL normal saline, and centrifuged. The supernatants are stored at −20° C. for drug analysis by HPLC.

TABLE 2

| | Time point | | | | | | | | | | | | | |
|---|---|---|---|---|---|---|---|---|---|---|---|---|---|---|
| Assessment | 0 h | 0.25 h | 0.5 h | 1 h | 3 h | 8 h | 1 d | 2 d | 3 d | 1 w | 2 w | 3 w | 4 w | 6 w |
| 1A. Joint tissue | X | | | | | | | | X | X | | X | | X |
| 1B. Plasma | X | X | X | X | X | X | X | X | X | X | | | | X |
| IBU conc. | | | | | | | | | | | | | | |
| 1C. Joint IBU | | | | | | X | X | | X | X | | | | X |
| conc. | | | | | | | | | | | | | | |
| 2. Therapeutic | | | | | | | X | X | X | X | X | X | X | |
| efficacy | | | | | | | | | | | | | | |

h, d, w = hours, days, weeks

The therapeutic efficacy of ILH can be determined by those skilled in the art, given the present description. For instance, rats that have received ILH intra-articular injection can be compared to those receiving oral IBU or no treatment (Groups 2D-F, respectively; Table 1). Rats are monitored according to the schedule in Table 2 to determine the therapeutic efficacy of the treatments using the following assessments:

Weight bearing—An incapacitance tester (Linton Instrumentation, Stoelting Co., Wood Dale, Ill.) is employed for determination of hind paw weight distribution using standard techniques.

Evaluation of Mechanical Hyperalgesia—The vocalization threshold of knee compression is measured using standard techniques.

Assessment of grip strength—Grip strength is assessed according to the methods available to those skilled in the relevant art.

Histological evaluation of the synovial inflammation—Rats are sacrificed at 6 weeks. The left knee joints are prepared and sections containing synovium, cartilage and bone are prepared. Sections are stained for cellularity with H&E and for proteoglycan content with safranin O. Synovial inflammation and cartilage degradation are evaluated by blinded histological evaluation of parapatellar synovium and knee articular cartilage, respectively. Villus hyperplasia, fibroblast proliferation, fibrosis, angiogenesis, mononuclear cell and polymorphonuclear cell infiltrations are graded as indicators of synovial inflammation. For cartilage degradation, surface erosion, proteoglycan content and chondrocyte necrosis are tested by conventional means.

1. A system for intraarticular drug delivery, comprising: a) a deliverable composition comprising a plurality of parts adapted to be mixed at the time of use in order to be delivered to an intraarticular space under conditions suitable to form a hydrogel in situ, b) one or more active agents included within the deliverable composition, and c) a device for mixing the composition parts, including active agent(s), in order to deliver the mixed composition to the intraarticular space. A system according to claim 1, wherein the plurality of parts comprises a first solution comprising between about 0.5% and about 3% weight per volume (w/v) carboxymethyl chitosan (CCN) in a first solvent, and a second solution comprising between about 0.5% and about 3% w/v oxidized carboxymethyl cellulose (OCMC) in a second solvent, and the first and second parts are mixed to form a single liquid phase that can be delivered to the joint space in order to form a hydrogel at the targeted joint site.

3. A system according to claim 1, wherein the bioactive agent comprises one or more NSAIDS.

4. A system according to claim 1, wherein the bioactive agent comprises ibuprofen.

5. A system according to claim 1, wherein the system, wherein the formed hydrogel is resorbable in situ.

6. A system according to claim 1, wherein the hydrogel is adapted to be resorbed for a period of weeks or longer.

7. A system according to claim 2, wherein the bioactive agent comprises One or more NSAIDS.

8. A system according to claim 7, wherein the bioactive agent comprises ibuprofen.

9. A system according to claim 8, wherein the system, wherein the formed hydrogel is resorbable in situ.

10. A system according to claim 9, wherein the hydrogel is adapted to be resorbed for a period of weeks or longer.

11. A method of intraarticular drug delivery, comprising: a) providing a deliverable composition comprising a plurality of parts adapted to be mixed at the time of use in order to be delivered to an intraarticular space under conditions suitable to form a hydrogel in situ, b) providing one or more active agents adapted to be included within the deliverable composition, and c) providing a device adapted to mix the composition parts, including active agent(s), d) mixing the plurality of parts to form a deliverable composition, and e) delivering the composition to the intraarticular space in a manner sufficient to form a hydrogel in situ.
